(19) United States
(12) Patent Application Publication
(10) Pub. No.: US 2007/0282166 A1
(43) Pub. Date: Dec. 6, 2007

(54) Endoscopic sleeve seal

(75) Inventors: Juan Carlos Ayala, Santiago (CL); Brian K. Rucker, King, NC (US)

Correspondence Address: BRINKS HOFER GILSON & LIONE/CHICAGO/COOK, PO BOX 10395, CHICAGO, IL, 60610, US

(73) Assignee: Wilson-Cook Medical Inc., Winston-Salem, NC (US)

(21) Appl. No.: 11/805,770

(22) Filed: May 24, 2007

Related U.S. Application Data
(60) Provisional application No. 60/810,387, filed on Jun. 1, 2006.

(51) Int. Cl. A61B 1/00

(52) U.S. Cl. 600/123; 600/154

(57) ABSTRACT

A medical sealing device is provided that substantially seals around one or more medical devices that are placed into the working channel of an endoscope; wherein the one or more medical devices have an irregular cross-sectional profile. When a sufficient back pressure or surface tension is present in the working channel, the sealing device collapses a portion of itself and conforms to a portion of the one or more medical devices present in the working channel, thus forming a seal around and between the one or more medical devices. The seal thereby substantially prevents the escape of fluids from the working channel.

### RELATED APPLICATIONS

The present patent document claims the benefit of the filing date under 35 U.S.C. §119(e) of Provisional U.S. Patent Application Ser. No. 60/810,387, filed Jun. 1, 2006, which is hereby incorporated by reference in its entirety.

### FIELD OF THE INVENTION

The invention relates to medical sealing devices, particularly those used in conjunction with endoscopes.

### BACKGROUND OF THE INVENTION

When an endoscope is used to view a passageway, such as the stomach, colon, intestine, bile duct, etc., a gas or liquid is often infused into the passageway to insufflate the passageway. Insufflating the passageway causes the passageway to expand, thus enabling the medical professional to better visualize and maneuver within the area in which the endoscopic procedure is being performed.

When a sufficient amount of fluid (including, but not limited to, gas, bile, blood, and radiopaque contrast fluid) is present in the passageway, back pressure occurs that may cause the fluid to leak out from the working channel of the endoscope; often times blood and bile puddle on the floor or on the person performing the procedure. In addition, surface tension also known as capillary-action can also cause fluid leakage even when no insufflation or backpressure is present. This is because the catheters and wire guides as well as the endoscope channels are small enough that they draw fluid out through the user end, even with no insufflation or backpressure. It is preferred that the fluid not leak from the endoscope while the endoscopic procedure is being performed, because such leakage may interfere with a successful outcome of the procedure and may contaminate the working area.

Seals are typically used to inhibit fluid leakage from the working channel of the endoscope. While traditional seals may seal around a single round-shaped device, most seals are incapable of inhibiting the escape of fluid from around one or more medical devices having a combined irregular cross-sectional profile that are disposed through the working channel of the endoscope. Furthermore, current seals often result in friction around the devices inserted therethrough. Friction inhibits the medical professional from using tactile feedback as a means for determining how the medical devices ought to be manipulated.

### BRIEF SUMMARY OF THE INVENTION

A seal device for use with an endoscope having a working channel extending from a working channel port is provided. The device includes a sleeve having a proximal portion, a distal portion, and a lumen extending therethrough. The proximal portion comprises an attachment mechanism that is adapted to attach to the working channel port of the endoscope, and the distal portion is adapted to conform around one or more medical devices that are inserted therethrough when subjected to a sufficient back pressure created by the proximal movement of fluids through the working channel. The one or more medical devices have an irregular cross sectional profile. The sleeve is configured for insertion into the working channel port of an endoscope.

Further, a seal device for use with an endoscope having a working channel extending from a working channel port is provided. The device includes a proximal portion, a distal portion, and a lumen extending therethrough. A diameter of the proximal portion is greater than a diameter of the distal portion. The sleeve is adapted to substantially seal around at least two medical devices inserted therethrough when a back pressure of about 20 mmHg exists within the working channel. The at least two medical devices have different diameters, and the at least two medical devices have a combined irregular cross-sectional profile. The sleeve creates minimal frictional force around the at least two medical devices inserted therethrough so as to not inhibit the movement thereof. The device further includes an attachment mechanism adapted to attach the sleeve to the working channel port of the endoscope.

Also provided is a method for sealing a working channel of an endoscope that extends from a working channel port of the endoscope. The method includes providing a seal sleeve wherein the seal sleeve is adapted to substantially seal around one or more medical devices inserted therethrough when a sufficient back pressure is present, wherein the one or more medical devices have an irregular cross-sectional profile. The method further includes attaching the seal sleeve to the working channel port of the endoscope with an attachment mechanism. The method further includes introducing the one or more medical devices through the seal sleeve, and providing a back pressure sufficient to cause the seal sleeve to conform to the one or more medical devices.

Still further, a seal device for use with an endoscope having a working channel extending from a working channel port is provided. The seal device includes a sleeve having a proximal portion, a distal portion, and a lumen extending therethrough. The proximal portion includes an attachment mechanism that is adapted to attach to the working channel port of the endoscope, and the distal portion is adapted to conform around one or more medical devices that are inserted therethrough when subjected to a sufficient back pressure or a surface tension created by the proximal movement of fluids through the working channel. The one or more medical devices have an irregular cross sectional profile. The sleeve is configured for insertion into the working channel port of an endoscope.

Still further, a seal device for use with an endoscope having a working channel extending from a working channel port is provided. The seal device includes a sleeve having a proximal portion, a distal portion, and a lumen extending therethrough. A diameter of the proximal portion is greater than a diameter of the distal portion. The sleeve is adapted to substantially seal around at least two medical devices inserted therethrough when a back pressure of about 20 mmHg or a surface tension exists within the working channel. The at least two medical devices have different diameters. The at least two medical devices have a combined irregular cross-sectional profile. The sleeve creates minimal frictional force around the at least two medical devices inserted therethrough so as to not inhibit the movement thereof. The device further includes an attachment mechanism adapted to attach the sleeve to the working channel port of the endoscope.

Still further, a method for sealing a working channel of an endoscope that extends from a working channel port of the endoscope is provided. The method includes providing a seal sleeve wherein the seal sleeve is adapted to substantially seal around one or more medical devices inserted therethrough when a sufficient back pressure or a surface tension is present, wherein the one or more medical devices have an irregular cross-sectional profile, attaching the seal sleeve to the working channel port of the endoscope with an attachment mechanism, introducing the one or more medical devices through the seal sleeve, and providing a back pressure or a surface tension sufficient to cause the seal sleeve to conform to the one or more medical devices.

### BRIEF DESCRIPTION OF THE SEVERAL VIEWS OF THE DRAWINGS

The embodiments will be further described in connection with the attached drawing figures. Throughout the specification, like reference numerals and letters refer to like elements. It is intended that the drawings included as a part of this specification be illustrative of the embodiments and should in no way be considered as a limitation on the scope of the invention.

FIG. 1 is a cross sectional view of a working channel of an endoscope having an embodiment depicted in an un-sealing state;

FIG. 2 is a cross sectional view of a working channel of an endoscope having the same embodiment as that depicted in FIG. 1 but here depicted in a sealing state;

FIG. 3 is a partial cross sectional view of a working channel of an endoscope having an embodiment of a seal sleeve and an attachment mechanism for attaching the seal sleeve to the working channel of the endoscope;

FIG. 4 is a partial cross sectional view of a working channel of an endoscope having an alternate embodiment of a seal sleeve and an attachment mechanism for attaching the seal sleeve to the working channel of the endoscope;

FIG. 5 is a perspective view of a working channel of an endoscope having an alternate embodiment of a seal sleeve and an attachment mechanism for attaching the seal sleeve to the working channel of the endoscope;

FIG. 6 is a perspective view of the circled portion 6 depicted in FIG. 5;

FIG. 7 is another perspective view of a working channel of an endoscope having an alternate embodiment of a seal sleeve and a mechanism for attaching the seal sleeve to the working channel of the endoscope;

FIG. 8 is a perspective view of an alternate embodiment of a seal sleeve and an attachment mechanism for attaching the seal sleeve to the working channel of the endoscope; and

FIG. 9 is a cross sectional view of a working channel of an endoscope having the same embodiment of a seal sleeve as that depicted in FIG. 8.

### DETAILED DESCRIPTION OF PRESENTLY PREFERRED EMBODIMENTS

The embodiments provide an apparatus that is able to maintain a seal around one or more medical devices having a combined irregular cross-sectional profile that are simultaneously inserted through the working channel of the endoscope while at the same time creating minimal or no frictional force.

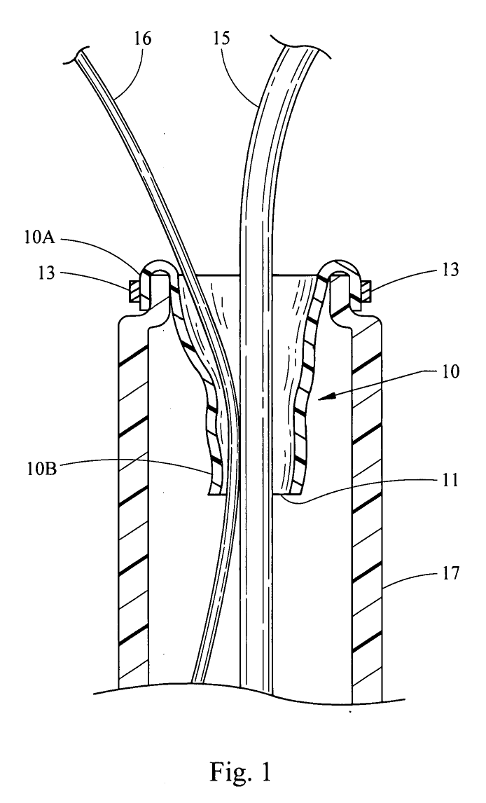

A more detailed description of the embodiments will now be given with reference to FIGS. 1-9. The present invention is not limited to those embodiments illustrated; it specifically contemplates other embodiments not illustrated but intended to be included in the claims.

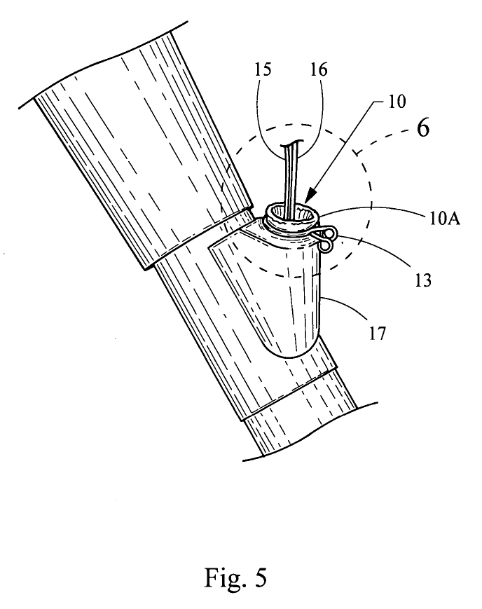

FIG. 1 is a cross sectional view of a working channel 17 of an endoscope containing seal sleeve 10 in an un-sealing state. Seal sleeve has a proximal portion 10A attached to the proximal end of working channel 17 via clamp 13. Clamp 13 is further depicted in FIGS. 5, 6, and 7. Distal portion 10B of seal sleeve 10 is generally not fixedly attached to working channel 17.

Seal sleeve 10 is made from a highly flexible, readily collapsible, substantially fluid-impermeable material having a low coefficient of friction, that will not degrade while in the presence of fluids within which it may come in contact. A preferred material is medical grade polyethylene; however seal sleeve 10 may be made from other materials that are flexible, readily collapsible, substantially fluid-impermeable, and that have a low coefficient of friction, including but not limited to, polyurethane, silicone, nylon, polyamides such as urethanes, polypropylene, polytetrafluoroethylene (PTFE), expanded polytetrafluoroethylene (ePTFE), and natural (latex) and polyisoprene rubbers. It is also contemplated that seal sleeve could be made from an elastic material that stretches when at least one medical device is placed therethrough, but then compresses and conforms around the medical device forming a substantially fluid-impermeable seal. Additionally, a removable stiffening insert may be added to seal sleeve 10 for easier placement of seal sleeve 10 within working channel 17.

The thickness of seal sleeve 10 is about 0.001-0.025 inches, although greater and lesser thicknesses are also contemplated depending on the type of material from which seal sleeve 10 is made. The material and thickness of the material from which seal sleeve 10 is made should be such that it is able to conform around one or more medical devices, having a combined irregular cross-sectional profile, inserted therethrough, without becoming permanently deformed or stretched.

A substantially fluid-impermeable seal is formed when a sufficient back pressure or surface tension is present. The back pressure or surface tension causes seal sleeve 10 to collapse and fold back onto itself and conform around the medical devices inserted therethrough. Additionally, the material should be sufficiently stiff so that seal sleeve 10 does not entirely invert into itself. Furthermore, the frictional force exerted by seal sleeve 10 onto a medical instrument inserted therethrough should be low enough so that tactile feedback at proximal portion 10B of seal sleeve 10 is preserved to allow the medical professional to obtain information from the distal end of the medical device inserted therethrough.

Wire guide 16 and catheter 15, collectively having an irregular cross-sectional profile, enter through proximal portion 10A of seal sleeve and exit through exit way 11 located at distal portion 10B of seal sleeve 10. Exit way 11 is large enough to allow medical devices to pass therethrough, but sufficiently small so as to be able to form a seal around each of the devices. Ideally, the diameter of exit way 11 is from about 0.020-0.200 inches, although other diameters are contemplated depending on the dimensions of the medical instrument inserted therethrough. Here, the inner diameter of exit way 11 is slightly larger than the combined outer diameters of the devices that will be placed through seal sleeve 10. Proximal portion 10A of seal sleeve is approximately about 0.157-0.197 inches wide, distal portion is approximately about 0.079-0.118 inches wide and seal sleeve 10 is approximately about 1 inch long; however, other dimensions are contemplated. Seal sleeve 10 is able to be configured so as to maintain a seal around one or more devices having various shapes and sizes that are inserted through working channel 17. Seal sleeve 10 is not limited to having a cone-shape; other shapes include but are not limited to a cylinder and duck-bill shape.

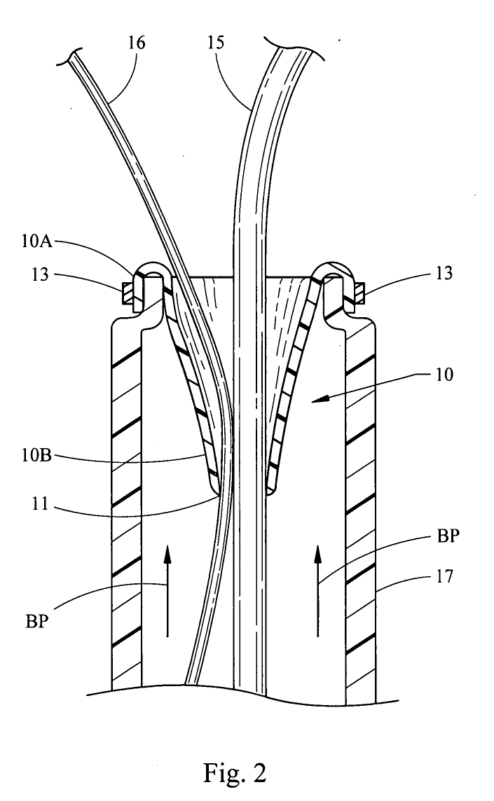

FIG. 2 depicts the embodiment of FIG. 1 but in a collapsed sealing state. Back pressure BP can occur naturally or as a result from insufflating an orifice. However, the embodiments disclosed herein do not require back pressure as the embodiments will also prevent leakage due to surface tension. Back pressure BP is generally up to about 20 mmHg and is such that it presses back up through working channel 17 in the proximal direction. Because seal sleeve 10 is made from a highly flexible, readily collapsible material, it will collapse onto itself in the proximal direction and conform around wire guide 16 and catheter 15 to form a substantially fluid-impermeable seal.

Because seal sleeve 10 is made from a material having a low coefficient of friction, the medical professional is able to use tactile feedback as a means for determining how wire guide 16 and catheter 15 ought to be further manipulated. Because a seal is maintained, the orifice remains insufflated and the area surrounding the working channel will not be contaminated by fluid escape.

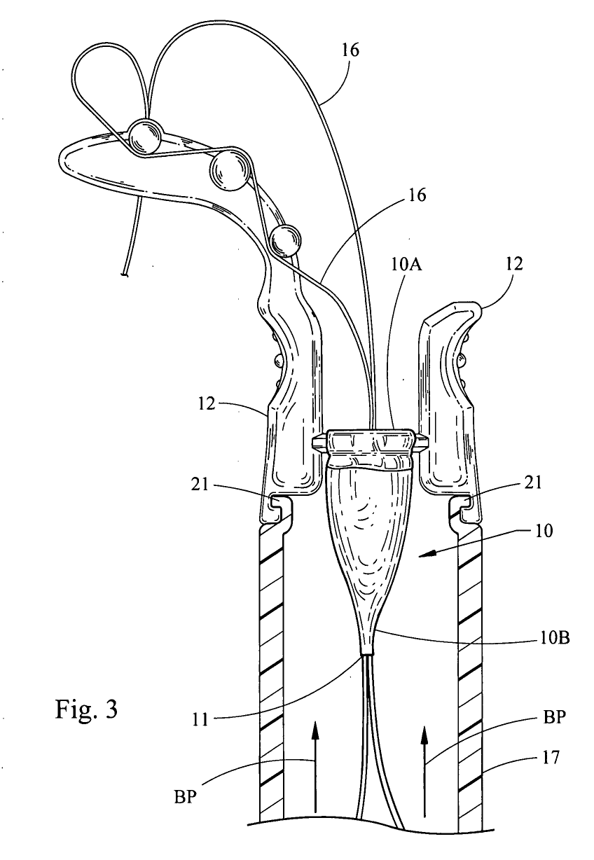

FIG. 3 depicts an embodiment of a seal sleeve 10 that is integrated into a Wire Guide Locking Device attachment mechanism, of the type produced by Cook Endoscopy. A wire guide locking device 12 is further described in U.S. patent application Ser. No. 11/350,483 that is hereby incorporated by reference. Seal sleeve 10 is attached to modified wire guide locking device 12 using a medically acceptable adhesive, however, other attachment mechanisms are contemplated. Wire guide locking device 12 attaches to working channel 17 having an outwardly extending flange 21.

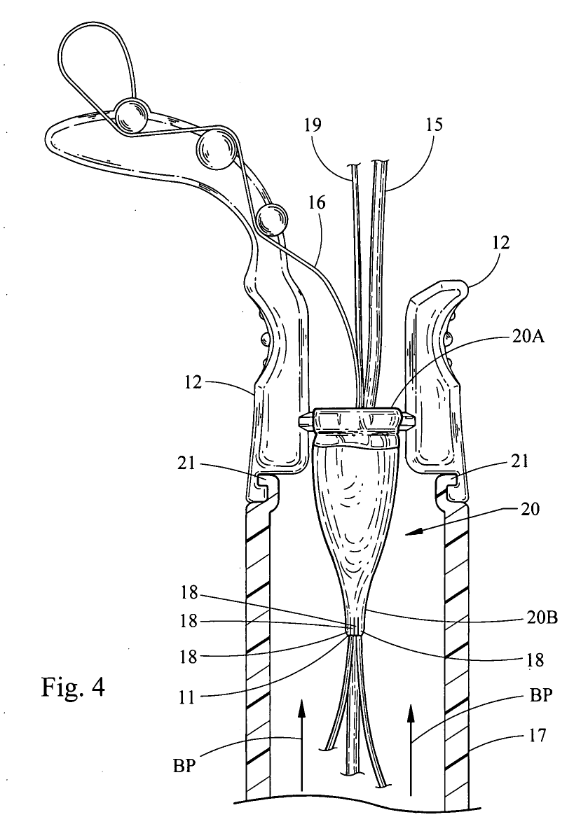

FIG. 4 depicts an alternate embodiment of seal sleeve 20, the proximal portion 20A of which is integrated into a modified wire guide locking device 12 as depicted in FIG. 3. Distal portion 20B of seal sleeve 20 includes a plurality of flaps 18. Flaps 18 help to create a seal around a plurality of medical devices 15, 16, 19, having a combined irregular cross-sectional profile, inserted through seal sleeve 20. In particular, when a sufficient back pressure or surface tension is present, a substantially fluid-impermeable seal is formed when flaps 18 fold onto themselves and conform around gaps that exist between the plurality of medical devices.

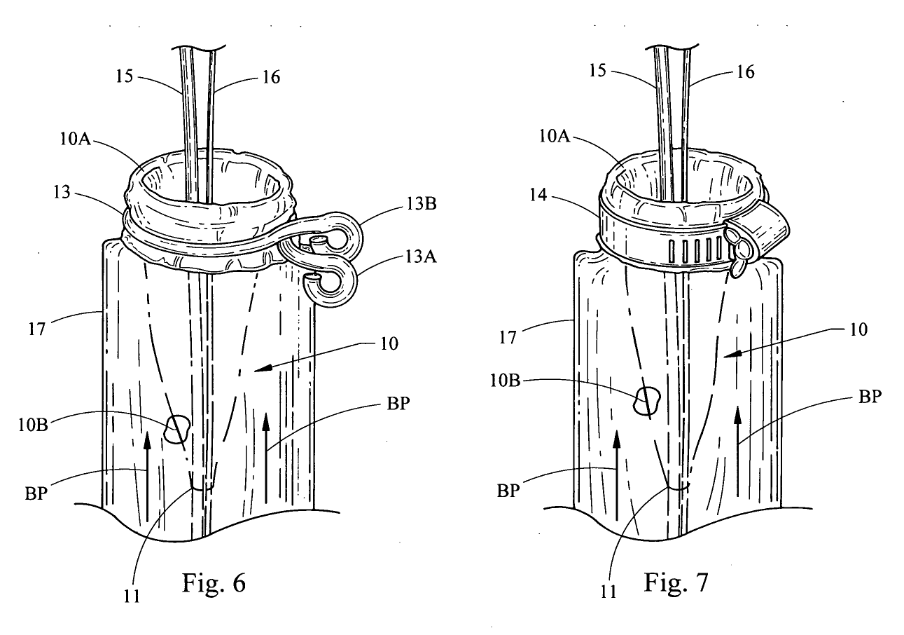

FIG. 5 depicts an embodiment of a seal sleeve 10 and a mechanism for attaching seal sleeve 10 to working channel 17 of endoscope, and FIG. 6 is a close-up view of the circled portion 6 depicted in FIG. 5. Seal sleeve 10 is placed into working channel 17 such that proximal portion 10A of seal sleeve 10 overlaps the end of working channel 17. Clamp arms 13A and 13B of clamp 13 are pressed towards each other causing the diameter of clamp 13 to expand. Clamp 13 is then placed around proximal portion 10A of seal sleeve 10 and clamp arms 13A and 13B are released causing clamp 13 to tighten and hold seal sleeve 10 firmly in place.

FIG. 7 depicts an alternate mechanism for attaching seal sleeve 10 to working channel 17 of endoscope. Here, seal sleeve 10 is placed into working channel 17 such that proximal portion 10A of seal sleeve 10 overlaps the end of working channel 17. Clamp 14 is placed around proximal portion 10A of seal sleeve 10 and is tightened so as to hold seal sleeve 10 firmly in place. Other attachment mechanisms are contemplated, including but not limited to, attaching seal sleeve 10 to working channel 17 using a medically acceptable adhesive, elastic, tape, or a cap.

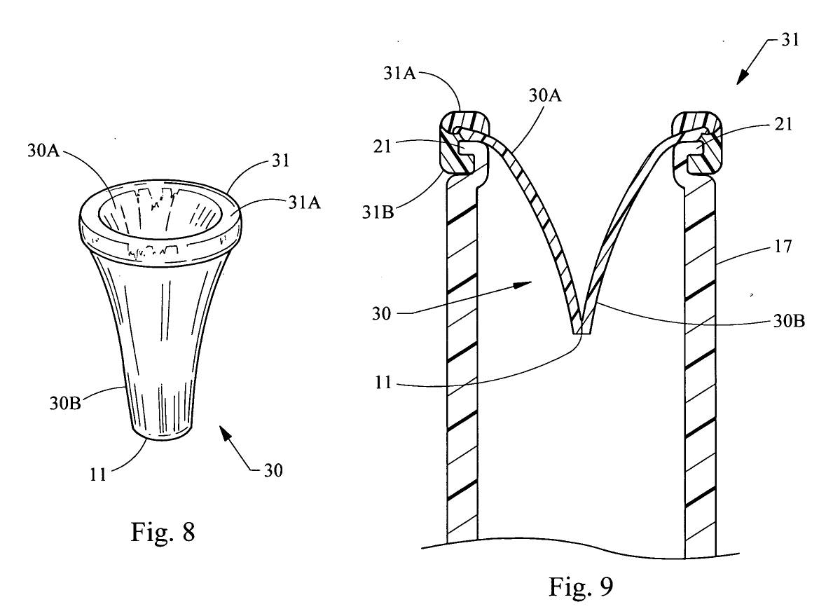

FIG. 8 depicts a perspective view of an alternate embodiment of seal sleeve 30; FIG. 9 depicts a cross sectional view of seal sleeve 30 disposed within working channel 17. Distal portion 30B of seal sleeve 30 comprises exit way 11. Proximal portion 30A of seal sleeve 30 is fixedly compressed between cap 31 that comprises two pieces 31A, 31B. Cap 31 removably attaches to outwardly extending flange 21 of working channel 17. It is also contemplated that cap 31 could be threaded to attach to a working channel having treads. Cap 31 could also be lined with a non-permanent adhesive substance so that cap 31 remains attached to a working channel lacking outwardly extending flange 21. Cap 31 can also be lined with a tacky material or further comprise a gasket to aid in attaching to a working channel lacking outwardly extending flange 21. Additionally, cap 31 can be configured in such a way that it snaps over a working channel lacking outwardly extending flange 21.

As is evident, the embodiments provide a very effective solution for maintaining a seal around one or more devices having an irregular cross-sectional profile that are simultaneously inserted into the working channel of an endoscope. The foregoing description and drawings are provided for illustrative purposes only and are not intended to limit the scope of the invention described herein or with regard to the details of its construction and manner of operation. It will be evident to one skilled in the art that modifications and variations may be made without departing from the spirit and scope of the invention. Changes in form and in the proportion of parts, as well as the substitution of equivalents, are contemplated as circumstances may suggest and render expedience; although specific terms have been employed, they are intended in a generic and descriptive sense only and not for the purpose of limiting the scope of the invention set forth in the following claims.

What is claimed is:

1. A seal device for use with an endoscope having a working channel extending from a working channel port, the seal device comprising:
a sleeve having a proximal portion, a distal portion, and a lumen extending therethrough;
wherein the proximal portion comprises an attachment mechanism that is adapted to attach to the working channel port of the endoscope, and the distal portion is adapted to conform around one or more medical devices that are inserted therethrough when subjected to a sufficient back pressure or a surface tension created by the proximal movement of fluids through the working channel;
wherein the one or more medical devices have an irregular cross sectional profile; and
wherein the sleeve is configured for insertion into the working channel port of an endoscope.

2. The seal device of claim 1 wherein the sleeve is made from a highly flexible, readily collapsible, substantially fluid-impermeable material having a low coefficient of friction, that will not degrade while in the presence of fluids within which it may come in contact.

3. The seal device of claim 1 wherein the sleeve is made from a material selected from the group consisting of polyethylene, polyurethane, silicone, nylon, polyamides, urethanes, polypropylene, polytetrafluoroethylene, expanded polytetrafluoroethylene, latex, polyisoprene rubbers, and an elastic material.

4. The seal device of claim 1 wherein the sleeve is made from a material that is about 0.001-0.025 inches thick.

5. The seal device of claim 1 wherein a diameter of the lumen located at the distal portion of the sleeve is slightly larger than a combined outer diameters of the one or more medical devices inserted therethrough.

6. The seal device of claim 1 wherein the distal portion further comprises a plurality of flaps.

7. The seal device of claim 1 wherein the sleeve shape is selected from the group consisting of a cone, cylindrical, or duck-bill.

8. The seal device of claim 1 wherein the back pressure is about 20 mmHg.

9. The seal device of claim 1 wherein the one or more medical devices inserted therethrough comprises a wire guide and a catheter.

10. The seal device of claim 1 wherein the one or more medical devices inserted therethrough have different diameters.

11. The seal device of claim 1 wherein the sleeve creates minimal frictional-force around the one or more medical devices inserted therethrough so as to not inhibit the movement thereof.

12. The seal device of claim 1 wherein the attachment mechanism is selected from the group consisting of a clamp, wire guide locking device, tape, elastic, adhesive, and a cap.

13. A seal device for use with an endoscope having a working channel extending from a working channel port, the seal device comprising:
a sleeve having a proximal portion, a distal portion, and a lumen extending therethrough;
wherein a diameter of the proximal portion is greater than a diameter of the distal portion;
wherein the sleeve is adapted to substantially seal around at least two medical devices inserted therethrough when a back pressure of about 20 mmHg or a surface tension exists within the working channel;
wherein the at least two medical devices have different diameters;
wherein the at least two medical devices have a combined irregular cross-sectional profile;
wherein the sleeve creates minimal frictional force around the at least two medical devices inserted therethrough so as to not inhibit the movement thereof; and
an attachment mechanism adapted to attach the sleeve to the working channel port of the endoscope.

14. The seal device of claim 13 wherein the attachment mechanism is selected from the group consisting of a clamp, wire guide locking device, tape, elastic, adhesive, and a cap.

15. A method for sealing a working channel of an endoscope that extends from a working channel port of the endoscope, the method comprising:
providing a seal sleeve wherein the seal sleeve is adapted to substantially seal around one or more medical devices inserted therethrough when a sufficient back pressure or a surface tension is present, wherein the one or more medical devices have an irregular cross-sectional profile,
attaching the seal sleeve to the working channel port of the endoscope with an attachment mechanism;
introducing the one or more medical devices through the seal sleeve; and
providing a back pressure or a surface tension sufficient to cause the seal sleeve to conform to the one or more medical devices.

16. The method of claim 15 wherein the back pressure introduced is about 20 mmHg.

17. The method of claim 15 wherein the attachment mechanism is selected from the group consisting of a clamp, wire guide locking device, tape, elastic, adhesive, and a cap.

18. The method of claim 15 wherein the one or more medical devices introduced comprises a wire guide and a catheter.

19. The method of claim 15 wherein the one or more medical devices have different diameters.

20. The method of claim 15 wherein the seal sleeve provided creates minimal frictional force around the one or more medical devices introduced so as to not inhibit the movement thereof.
